(19) United States
(12) Patent Application Publication
(10) Pub. No.: US 2014/0364717 A1
(43) Pub. Date: Dec. 11, 2014

(54) METHOD AND DEVICE FOR CONTINUOUS MEASUREMENT OF INTRAOCULAR PRESSURES

(71) Applicant: IMPLANDATA OPHTHALMIC PRODUCTS GMBH, Hannover (DE)

(72) Inventors: Max Ostermeier, Seevetal (DE); Stefan Meyer, Hannover (DE)

(73) Assignee: IMPLANDATA OPHTHALMIC PRODUCTS GMBH, Hannover (DE)

(21) Appl. No.: 14/369,728

(22) PCT Filed: Jan. 17, 2013
(86) PCT No.: PCT/EP2013/050797

§ 371 Date: Jun. 30, 2014

(30) Foreign Application Priority Data
Jan. 19, 2012 (DE) 10 2012 100 441.2

(51) Int. Cl. A61B 3/16; G06F 19/00; A61B 5/00

(52) U.S. Cl. CPC A61B 3/16; A61B 5/4833; G06F 19/3456
USPC 600/398

(57) ABSTRACT

A method to obtain and view lop time developments of a patient and to generate database relating to a patient's individual IOP development, includes the steps of continuous measurement and storage of IOP data of a patient over a period of time of at least 24 hours, during a normal day and without medication, and then continuous measurement and storage of IOP data of a patient over a period of time of at least 24 hours, the patient taking their medication, wherein medication times, medication durations, doses, active substances, events throughout the patient's day are recorded, wherein the IOP data are measured using at least double the frequency of an assumed time-based pattern in the IOP development and wherein the stored data are relayed to an analysis unit and the data is analysed.

The present invention relates to a method and to a device for continuous measurement of intraocular pressures (IOP).

Glaucoma is a term describing a number of ocular diseases which have various causes and all of which lead to a loss of nerve fibres. The result is a characteristic loss of the field of vision (scotoma) and in extreme cases blindness of the eye. A high IOP value is considered to be a high risk factor for glaucoma. The temporal development of the IOP values depends heavily on the patient. In terms of their general efficacy, latency and duration of action, the effect of IOP-lowering drugs also largely depends on the patient. Owing to these two factors that are dependent on the patient, the intraocular pressure is often not lowered by a sufficient amount, and therefore extensive damage to the optic nerve culminating in blindness of the patient cannot be ruled out despite the medication.

In light of this, measurements have been proposed for determining an individual IOP profile over a relatively long period of time. Up to now, these have taken place after a patient has been admitted to a clinic. The measurement intervals are typically longer than two hours, contingent on organisational factors and the requirement for topical anaesthesia. This achieves only a rudimentary data density. One drawback is that the patient has to be woken up for nocturnal measurements, and this has unknown effects on the patient's IOP profile. Lastly, in some cases different measuring devices are also used for daytime and night-time measurements, with only secondary variables generally being measured, since direct manometry of the IOP is never used by default, and therefore there can be no effective treatment resulting from the data thus obtained.

In order to avoid some of these drawbacks, DE 10 2004 056 757 Al proposed using an implantable, extrascleral measurement apparatus comprising a capacitive pressure sensor along with a suitable electronics; DE 10 201 0 035 294 of the applicant proposed a measurement system which has a pressure-transferring, dimensionally-stable resilient housing for biocompatible contact with the sclera of the eye and in which are embedded pressure sensor means having at least one clear pressure sensor surface.

Methods are fundamentally known which record the medical data of a patient and transmit this to remote receivers for storage and further processing. One example of this is U.S. 6,669,631 A1, according to which an implanted medical measurement sensor transmits biological data to a remote receiver, which stores these data in a centralised database, which in turn contains statistical data from public databases and matches the patient's data with the statistical data using data mining techniques. The intention behind this method is to specify in each case a particular therapeutic plan, treatment plan, treatment progress report or usage rules, and to automatically generate reports and warnings. U.S. 6,742,895 A1 discloses a device and method for diagnosing and treating glaucoma patients. The device contains a software program which can be accessed via the Internet and contains a menu-driven data interpretation module. The option of accessing an online reference library is also provided. A report module generates patient-specific reports relating to glaucoma diagnosis, treatment and analysis.

The drawback of these known systems is that they do not ensure sufficient data security and data density.

It is therefore an object of the invention to provide a method and a device which generate an optimised database relating to a patient's individual IOP development.

The object in terms of the method is achieved with a method for obtaining the development over time of a patient's IOP, in that the method comprises the following steps:

a) continuous measurement and storage of IOP data of a patient over a period of time of at least 24 hours, during a normal day and without medication, and then

b) continuous measurement and storage of IOP data of a patient over a period of time of at least 24 hours, the patient taking their medication, wherein

c) medication times, medication durations, doses, active substances, events throughout the patient's day are recorded, wherein

d) the IOP data are preferably measured using at least double the frequency of an assumed time-based pattern in the IOP development and wherein

e) the stored data are relayed to an analysis unit and the data is evaluated.

The method according to the invention advantageously proposes quasi-continuous measurement of the IOP development of a patient during a normal day, wherein the method begins by determining the baseline IOP development without medication over at least 24 hours, in order to thus detect the cyclical fluctuations of the pressure development. Normally, 24 hours are sufficient for this, although a longer measurement period is also in line with the invention, since individual progressions that have a rhythm of more than 24 hours are conceivable. Next, the IOP development of a patient taking medication is measured, the medication times, durations, doses, the active substance applied and events throughout the patient's day being recorded together with the measurement data. According to the invention, this serves to establish and quantify possible environmental influences on the IOP development. In this case, the measurement frequency follows in particular the Nyquist-Shannon or the Whittaker-Kotelnikow-Shannon sampling theorem in order to minimise aliasing artifacts, in other words at at least double the frequency of an assumed time-based pattern in the IOP development. According to the invention, the data thus acquired are relayed to an analysis unit and evaluated here, in particular evaluated automatically, so as to increase the quality thereof and to achieve a meaningful database that contains the individual situation of a patient in physiological, psychological and environmental terms.

In the embodiment of the method, it is provided that in step b) a first drug is administered at a first time and a second drug is administered at a second time and additional drugs are possibly administered at other times, wherein the IOP data of the patient are continuously measured and stored after each time over a period of at least 24 hours, wherein the period of time between two successive times is selected such that the duration of action of the drug administered first has at least nearly ended. This embodiment advantageously allows for precise determination of an individual profile of action of an active substance. In this case, step b) of the method is repeated n times, n being the number of the active substances, active substance combinations or active substance dosages to be tested. In this regard, a drug can also be administered at a plurality of times or alternately with another drug.

Particularly advantageous is the embodiment of the method according to which two or more active substances are administered at one time in step b). This also allows the individual IOP development to be established with combined active substances. According to the invention, there is also a variant in which the period between the two times at which two active substances are given is significantly shorter than 24 hours, in other words the times are closer together. Depending on the individual active development, the later active substance can for example be given after 6 hours if the effect of the active substance given previously has already diminished.

Particularly advantageous is one development of the invention according to which information and/or treatment instructions are communicated to the patient during the implementation of the method. A great number of advantages are achieved thereby. Specifically, the patient is not left alone during the measurement but is kept informed about the current step and given guidance as to how it will be carried out. The patient is also reminded of an imminent step and the patient's questions regarding the progress of the method can be answered in a timely manner so that a particularly high quality, detailed database is achieved.

The evaluative analysis of the measurement data in step e) also takes into account the target pressure of the patient, their personal preferences, active substance tolerances and the dosage levels and times, so that the doctor in charge is provided with a particularly well analysed database that is reliable and meaningful in all senses. This also relates to the provision of statistical parameters by the evaluative data analysis, such as mean values, medians, standard deviations, 3δ values, FFT and so on.

Repeating steps a) and b) at different times for a complete medicinal control of a patient in order to provide the doctor with data regarding the success of the therapy or lack thereof is also a part of the invention.

The object in terms of the device is achieved by the combination of features of claim 8, wherein the device for acquiring and evaluating medical data comprises at least one measuring device and an analysis unit, wherein the at least one measuring device comprises a data acquisition unit having at least one sensor, at least one data store, at least one data transmission apparatus and at least one operation and communication interface, wherein the analysis unit comprises a data transmission apparatus and a processing unit, wherein the processing unit is designed to apply data analysis and structure-testing statistical algorithms and filter methods to the measurement data and wherein the processing unit comprises a display unit.

Developments of the device according to the invention are provided in the dependent claims.

The invention is described by way of example in a preferred embodiment with reference to the drawings, further advantageous details being inferable from the figures of the drawings.

Parts which share the same function are given the same reference numeral.

In the drawings:

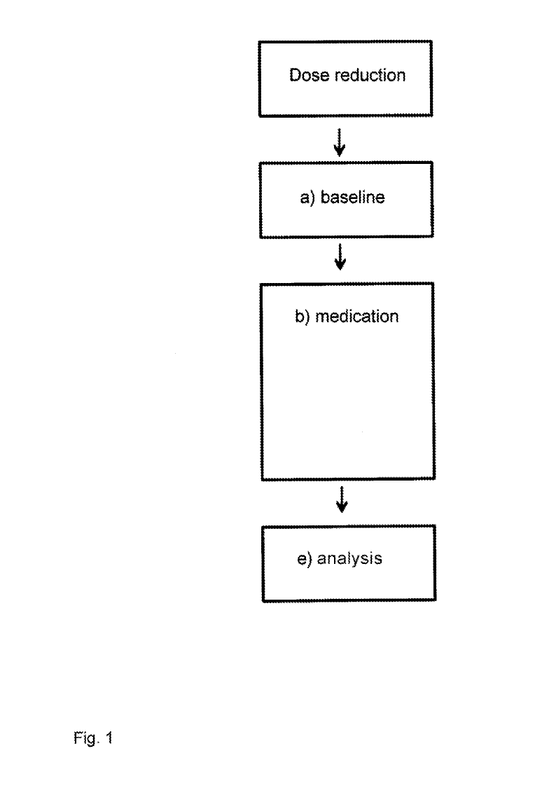

FIG. 1 is a flow chart of the method,

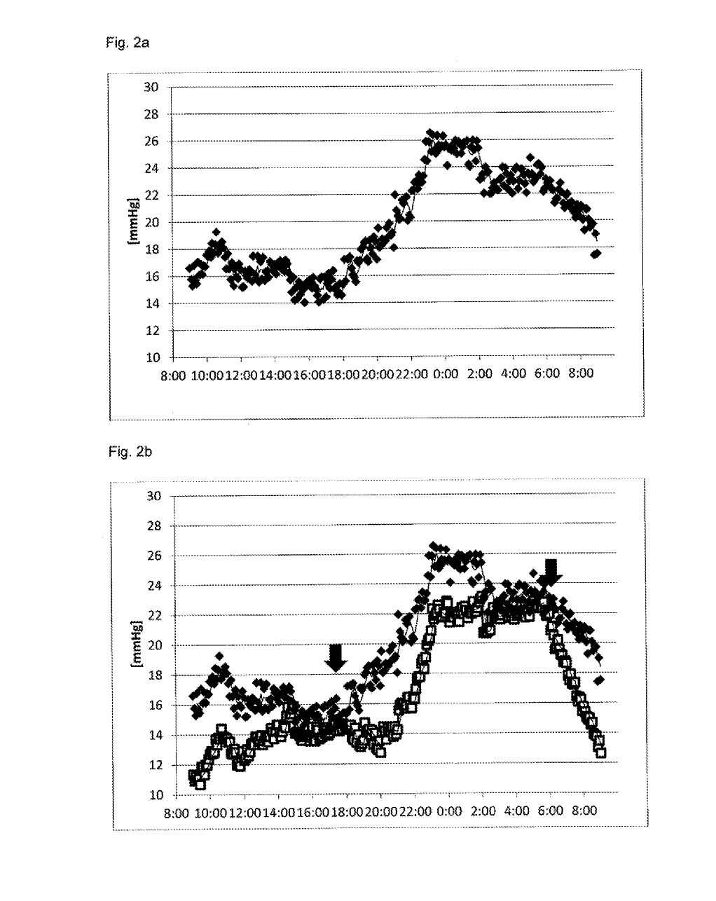

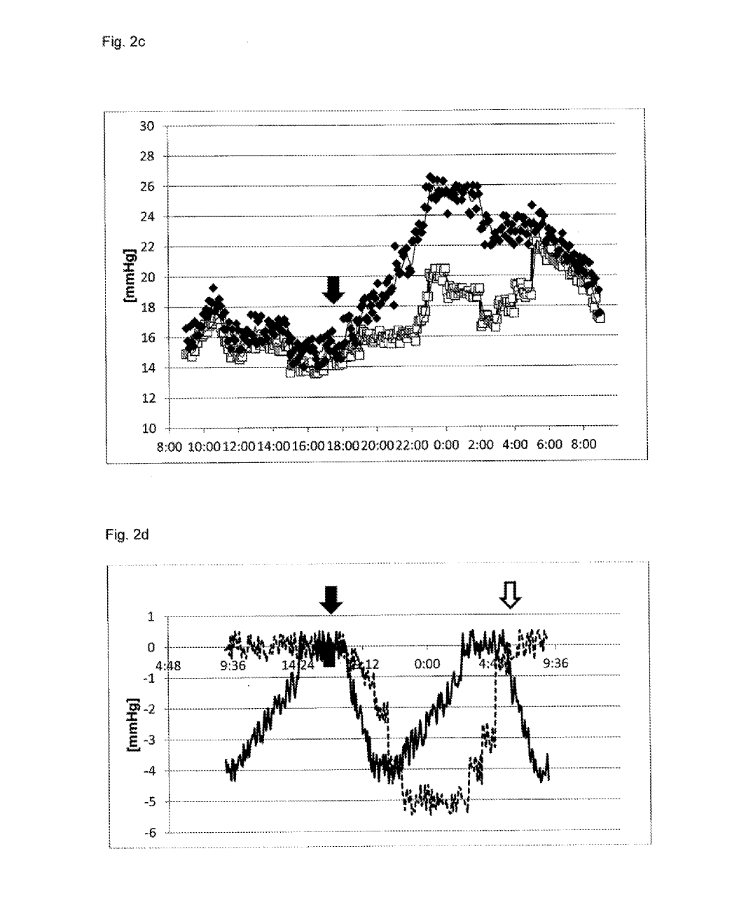

FIG. 2a-d are IOP development models,

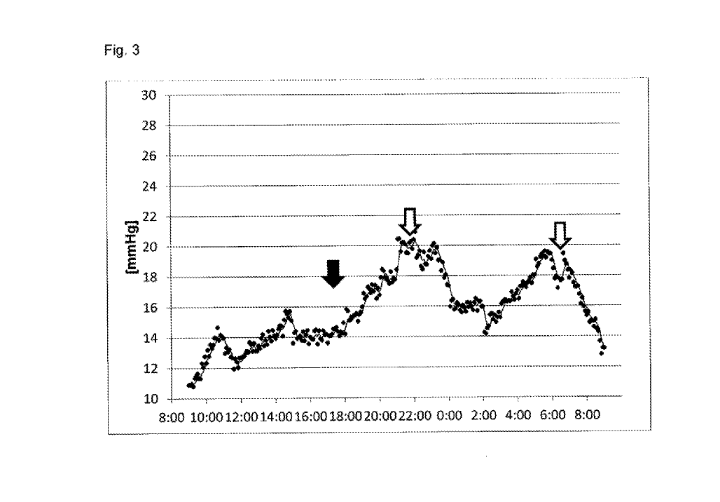

FIG. 3 shows the IOP development of a follow-up examination, and

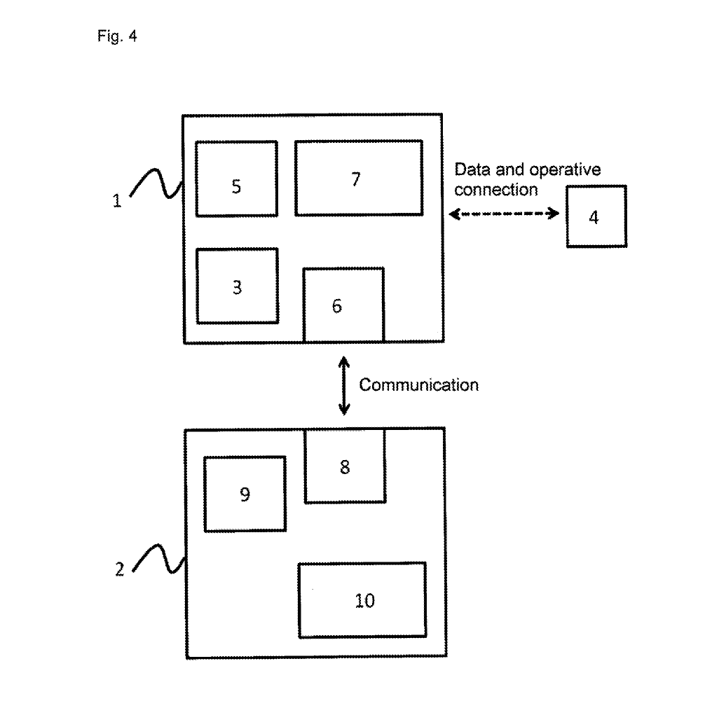

FIG. 4 is an outline view of a device according to the invention.

FIG. 1 shows a schematic flow chart of the method according to the invention. This begins with the baseline IOP of a patient being measured, in preparation for which the patient has to reduce the dose of their current medication, possibly for a few days. Their IOP baseline curve is then recorded for at least 24 hours. The measurement frequency in this case in 0.003 Hz, in other words one measurement every 5 minutes. The measurement frequency can be higher or lower, although a measurement frequency of 5 minutes is considered to be continuous according to the invention. The frequency has to be selected to be so high that the data are meaningful in terms of time dependencies of the IOP. It is imperative that the measurement is carried out in the normal living environment of the patient and that the patient themselves implements or begins the measurement. According to the invention, physical support by a doctor or assistance staff is avoided so that the temporal development of the IOP values is not influenced. In this regard, a sensor is used which is temporarily implanted in the patient, for example the sensor described by the applicant in DE 10 2010 035 294. Along with this one sensor for the IOP, the method can also use additional sensors, for example pulse or blood pressure sensors. Once the baseline has been established, a first drug is administered at a first time, whereby a new measurement cycle of at least 24 hours is initiated. Which active substance is being administered at which time and in which dosage is communicated to the patient by the measuring device 1. The patient carries out the appropriate instructions, the measuring device electronically providing the corresponding data and times with a time stamp and storing said data and times. Particular events, such as physical or mental stresses, meals, etc., are also stored with a time stamp by the measuring device. Alternatively, the patient can also keep a non-electronic diary, although the electronic data storage is preferred since the data are more easily available. According to the invention, this first active measurement can be followed by additional active measurements (see FIG. 2a-e in this regard). The data established thus are stored in the measuring device and passed to an analysis unit which filters the data, in order to eliminate noise, and performs a statistical and analytical evaluation thereof. In this regard, the IOP developments are shown graphically, it also being possible for OPA (ocular pulse amplitude) views to be provided too. In the graphical evaluations, envelopes for local maximums and minimums can be provided. The analysis unit also generates overviews in the form of tables which may contain for example daily fluctuation ranges, compliance data of the patient (rate of measurements carried out versus the possible number of measurements) or temporal patterns or other types of patterns that can be identified.

FIGS. 2a to 2d show IOP development models over the course of a method having more than one medication time. The views are in fact similar to those generated by the analysis unit.

FIG. 2a shows a baseline IOP profile following a prior dosage reduction of a medication. Twenty-four hours are shown on the x-axis, starting at 8 am on one day and ending at 8 am on the next day. The y-axis shows the measured IOP values in mmHg. It can be seen that this patient has high IOP values of over 21 mmHg predominantly at night.

FIG. 2b shows an evaluation, similar to how it can be obtained following implementation of step b) of the method, the two sets of data being normalised one after the other. Following step a), a first drug A was administered in step b) at two times that were less than 24 hours apart, namely at a first time, 6 pm, at a first dosage and then at a second time, 6 am, at a second dosage identical to the first, wherein the measurement was carried out from 6 pm to 6 pm the following day. Here, the evaluation consisted in temporal normalisation of the IOP development of the medication to that of the baseline measurement. The upper curve shows the baseline from step a), the lower curve the individual profile of action of the first drug A. The two arrows indicate the two aforementioned medication times. It can be seen that using drug A significantly reduces the IOP values over practically the entire day. It can further be seen that the reduction is not sufficient to allow the IOP to fall below 21 mmHg at night.

FIG. 2c shows the evaluation of a step b) of the method, in which a second drug B is administered at a time (6 pm) and the ensuing IOP developments were measured over 24 hours. This was carried out following the measurement according to FIG. 2b. Here, the evaluation also consisted in temporal normalisation, as described above. The upper curve is the baseline, the lower curve the medication line. It can be seen that the IOP is dramatically reduced during the night, the IOP falling below 21 mmHg for practically the whole 24 hour measurement period. This threshold was only slightly exceeded in the early morning.

According to the invention, the processes shown in step b) can be repeated on successive days or on days with intervals in between, for example in order to determine the IOP over the course of a week.

The evaluation according to step e) is also carried out according to FIG. 2d, in which the reductions in the IOP induced by the respective drugs in the respective manners of application are shown normalised to one another. The y-axis shows the change in the IOP in mmHg compared with the baseline, the x-axis contains the time development. In this evaluation, the latency, duration of action and strength of action of a medication can be seen clearly.

These data are provided to a doctor, who can work out therefrom individually determined and thus individually effective therapy suggestions which take into account the individual requirements of the patient, such as undisturbed rest at night, few drugs, and convenient administration times, together with the desired target pressures or average pressure values. A recommendation of this type can even take place automatically, provided that the boundary conditions are input.

FIG. 3 shows the IOP development of a follow-up examination in the course of a therapy that has already been drawn up. This repetition of the measurement according to the invention over 24 hours in the normal daily environment of the patient with all relevant events being recorded allows it to be established whether the therapy currently being used is still effective or if a correction is necessary. In the latter case, the method steps described above have to be repeated, starting with the dosage reduction of the current medication. The arrows in FIG. 3 again indicate the medication times of the two different drugs A and B. It can be seen that the IOP is still below 21 mmHg, and therefore the doctor does not have to recommend a new therapy.

The evaluation apparatus gives the doctor a summary of the measurements in the form of a report in the views that the doctor has specified, such as time series, tabular overviews, waterfall charts and the like.

FIG. 4 shows an outline view of a device according to the invention, which comprises at least one measuring device 1 carried around by the patient. The device is designed according to the invention such that a plurality of measuring devices 1 can be operated at the same time. The measuring devices 1 receive data from a sensor 4 belonging to the measuring device 1. The sensor 4 is for example a pressure sensor, as used by the applicant, although the sensor 4 can also consist of a plurality of sensors which alongside the IOP also pick up blood pressure, heart rate or other medically relevant data and relay this data to the measuring device 1. The measuring device has a sufficiently large data store 5 to record the measurement data of the one or more sensors 4 at a sufficient temporal resolution over relatively long periods of time. The measuring device 1 also has a data transmission apparatus 6 which relays the stored data to an analysis unit 2. The data transmission apparatus 6 is designed according to the invention such that it transmits the data in a wired or wireless manner, for example being formed as a WLAN, WWAN, Bluetooth, IR interface, plug contact or the like. The measuring device 1 further comprises an operation and communication interface 7. The operation interface is for example a screen/keyboard combination, or a touchscreen, although a voice control can also be provided. The option for manual data input is provided according to the invention. The communication interface is used for the communication between the patient and a carer, it also being possible for the carer to be a computer program that gives out appropriate instructions. This communication interface 7 displays treatment instructions or alarms or questions to be answered to the patient and is designed such that the patient can communicate information to the carer. For example, the communication interfaces could also comprise a separate LED alarm, which visually displays relevant alarms by blinking or lighting up. The communication interface could also comprise a corresponding loudspeaker. The communication between the carer and the patient can also take place via a smartphone app or a text message, in other words by means of such devices available to the patient. In this case, the communication interface would be taken out of the measuring device 1. The communication interface also allows the patient to be monitored and alarms to be transmitted using alarm limits and alarm algorithms that can be individually configured by the doctor in charge. This advantageously ensures that the individual target IOP for each patient is taken into account, so that IOP values are not exceeded to critical levels. The number of erroneous alarms is thereby also kept low, for example by selecting different alarm limits for night and day. It is also possible to configure how many times a pressure increase above the maximum pressure can be tolerated in each individual case. According to the invention, the alarms of the analysis unit 2 are received by the communication interface 7, but are kept from the patient. In this case, the alarm algorithms can be locally defined and carried out on the measuring device 1 or on a central support program.

According to the invention, the analysis unit 2 can be arranged either in the measuring device 1 or spatially remote therefrom, but in any case it comprises a data transmission apparatus 8 in order to receive data from the measuring device(s) 1 and to communicate therewith. If the analysis unit 2 is arranged in the measuring device 1, the individual patient data received by the one of more sensors 4 can be evaluated in the measuring device 1 itself and can be communicated to a doctor in charge as a report or can be rejected or read out by said doctor. However, the embodiment in which the analysis unit 2 is spatially separated from the measuring device 1 under operating conditions is more advantageous. In this case, according to the invention the analysis unit 2 is a local or a central database or a cloud-based program, in other words either a computer system provided in the doctor's practice or a computer system that can be accessed via the Internet or in another manner, for example in a computing centre or a cloud-based system.

This analysis unit 2 comprises a processing unit 9 and a display unit 10, the display unit being a monitor for example. The processing unit 9 filters the data in order to eliminate noise and other disturbance signals, and is designed to apply data analysis methods and structure-checking statistical algorithms, such as ANOVA, FFT, Welch's method, Lomb-Scargle periodogram, curve superposition, least square fit, heuristic search algorithms and data mining methods. In particular, the processing unit 9 is designed to algorithmically identify problematic high IOP phases in the time development studied, and to break down the established individual medication profiles of action into characteristics such as latency, efficacy and duration of action.

The method according to the invention significantly improves the data acquisition, which allows for a significant improvement in the data analysis. In each case, is it specific to the patient and can be tailored to their requirements. As a result, a doctor is provided with meaningful data in order to be able to draw up a therapy and to readjust this early on in the event of a change in the IOP developments of the patient or the patient's response.

### LIST OF REFERENCE NUMERALS

1 measuring device

2 analysis unit

3 data acquisition unit

4 sensor

5 data store

6 data transmission apparatus

7 operation and communication interface

8 data transmission apparatus

9 processing unit

10 display unit

1. Method for obtaining and viewing IOP time developments of a patient, comprising the steps
a) continuous measurement and storage of IOP data of a patient over a period of time of at least 24 hours, during a normal day and without medication, and then
b) continuous measurement and storage of IOP data of a patient over a period of time of at least 24 hours, the patient taking their medication, wherein
c) medication times, medication durations, doses, active substances, events throughout the patient's day are recorded, wherein
d) the stored data are relayed to an analysis unit and the data are analysed.

2. Method according to claim 1, wherein in step b) a first drug is administered at a first time and a second drug is administered at a second time and additional drugs are possibly administered at other times, wherein the IOP data of the patient are continuously measured and stored after each time over a period of at least 24 hours, wherein the period of time between two successive times is selected such that the duration of action of the drug administered first has at least nearly ended.

3. Method according to claim 1, wherein two or more active substances are administered at one time in the step.

4. Method according to claim 1, in which information and/or treatment instructions are communicated to the patient.

5. Method according to claim 1, in which in step e) the measurement data are analysed taking the following factors into account: the target pressure of the patient, the personal preferences of the patient, active substance tolerances, optimum active substances, optimum dosages and dosage times.

6. Method according to claim 6, in which statistical parameters are issued in step e).

7. Method according to claim 1, in which an efficacy control is carried out, in particular is repeated, at a different time to the first implementation of the method.

8. Device for carrying out a method according to claim 1 for acquiring and viewing IOP data, comprising at least one measuring device (1) and an analysis unit (2),
wherein the at least one measuring device (1) comprises a data acquisition unit (3) having at least one sensor (4), at least one data store (5), at least one data transmission apparatus (6) and at least one operation and communication interface (7),
wherein the analysis unit (2) is a local database and comprises a data transmission apparatus (8) and a processing unit (9), wherein the processing unit is designed to apply data analysis and structure-testing statistical algorithms and filter methods to the measurement data and wherein the processing unit (9) comprises a display unit (10).

9. Device according to claim 9, characterised in that the analysis unit (2) is spatially remote from the data acquisition unit (1).

10. Device according to claim 9, characterised in that the display unit (10) is spatially remote from the analysis unit (2).
